(19) United States
(12) Patent Application Publication
(10) Pub. No.: US 2025/0095790 A1
(43) Pub. Date: Mar. 20, 2025

(54) METHOD, INFORMATION PROCESSING DEVICE, AND RECORDING MEDIUM FOR PERFORMING PREDICTION RELATED TO POLYCONDENSATION REACTION

(71) Applicant: DIC Corporation, Tokyo (JP)

(72) Inventors: Atsushi Nagao, Osaka (JP); Yoshimasa Matsufuji, Osaka (JP); Naoki Shibamoto, Osaka (JP); Shinsuke Kariura, Tokyo (JP); Nobuaki Bessho, Tokyo (JP)

(73) Assignee: DIC Corporation, Tokyo (JP)

(21) Appl. No.: 18/566,760

(22) PCT Filed: May 22, 2023
(86) PCT No.: PCT/JP2023/019013

§ 371 Date: Dec. 4, 2023

(30) Foreign Application Priority Data
Feb. 24, 2023 (JP) 2023-027827

(51) Int. Cl. G16C 20/10; G16C 20/70; G16C 20/80

(52) U.S. Cl. CPC G16C 20/10; G16C 20/70; G16C 20/80

(57) ABSTRACT

A method for performing prediction related to a polycondensation reaction is executed by an information processing device, and includes: training a prediction model based on actual data including a plurality of explanatory factors and an objective factor that are related to the polycondensation reaction; and predicting, with the prediction model, the objective factor during the polycondensation reaction based on the explanatory factors related to the polycondensation reaction. The explanatory factors include a plurality of feature values obtained by a clustering analysis of time-series data from a plurality of measurement instruments at a dehydration temperature rising process, and the objective factor includes at least either a viscosity or an acid value.

### CROSS-REFERENCE TO RELATED APPLICATIONS

The present application claims priority from Japanese Patent Application No. 2023-027827, filed on Feb. 24, 2023, the contents of which are incorporated herein by reference.

### BACKGROUND

### Technical Field

The present disclosure relates to a method for performing prediction related to a polycondensation reaction, an information processing device, and a recording medium storing instructions.

### Description of Related Art

Conventionally, methods for performing prediction related to chemical reactions have been developed (for example, PTL 1).

### PATENT LITERATURE

PTL 1: WO 2003/026791

The technique described in PTL 1 has described the use of modeling techniques such as neural networks, partial least squares, and principal component regression to optimize the control of reactor systems. However, specific design methods and optimization in performing predictions related to polycondensation reactions have not been considered, and there has been room for improvement in the prediction technology related to the polycondensation reactions.

### SUMMARY

One or more embodiments of the present disclosure made in view of such circumstances improve the prediction technology related to the polycondensation reactions.

(1) A method in one or more embodiments of the present disclosure is a method for performing prediction related to a polycondensation reaction executed by an information processing device, the method comprising:
a step of training a prediction model based on actual data comprising a plurality of explanatory factors and an objective factor that are related to the polycondensation reaction; and
a step of predicting, with the prediction model, the objective factor during the polycondensation reaction based on the explanatory factors related to the polycondensation reaction, in which
the explanatory factors include a plurality of feature values obtained by a clustering analysis of time-series data from a plurality of measurement instruments at a dehydration temperature rising process, and
the objective factor includes at least either a viscosity or an acid value.

(2) The method in one or more embodiments of the present disclosure is the method according to (1), in which the explanatory factors include a theoretical value in a reaction physics model.

(3) The method in one or more embodiments of the present disclosure is the method according to (1) or (2), in which the polycondensation reaction is a dehydration-condensation reaction of a polyester.

(4) The method in one or more embodiments of the present disclosure is the method according to any one of (1) to (3), in which
in the step of predicting, a change over time (i.e., time-course change) of the objective factor during the polycondensation reaction is previously predicted, and
the explanatory factors include a cumulative calculation value of added raw material during the polycondensation reaction and a raw material addition time, and
the method further comprises: changing the cumulative calculation value and the raw material addition time, as a part of a condition for calculating a predicted value of the change over time of the objective factor, and outputting a visualization graph illustrating a relationship among a reaction end time, an amount of the added raw material, and the raw material addition time.

(5) The method in one or more embodiments of the present disclosure is the method according to (4), in which the visualization graph is a heat map or a contour map in which a first axis indicates the raw material addition time and a second axis indicates the amount of the added raw material.

(6) The method in one or more embodiments of the present disclosure is the method according to (4) or (5), in which the visualization graph includes plots representing the actual data.

(7) The method in one or more embodiments of the present disclosure is the method according to any one of (1) to (6), in which the prediction model is a neural network model including: an input layer; an intermediate layer; and an output layer, and a coefficient of an activation function of the intermediate layer is larger than a coefficient of an activation function of the output layer.

(8) An information processing device in one or more embodiments of the present disclosure is an information processing device performing prediction related to a polycondensation reaction, the information processing device comprising: a control unit that:
trains a prediction model based on actual data comprising a plurality of explanatory factors and an objective factor related to the polycondensation reaction, and
predicts, with the prediction model, the objective factor during the polycondensation reaction based on the explanatory factors that are related to the polycondensation reaction,
the explanatory factors include a plurality of feature values obtained by a clustering analysis of time-series data from a plurality of measurement instruments at a dehydration temperature rising process, and
the objective factor includes at least either a viscosity or an acid value.

(9) A non-transitory computer-readable recording medium according to one or more embodiments of the present disclosure is a non-transitory computer-readable recording medium storing instructions performing prediction related to a polycondensation reaction by an information processing device that comprises a processor, the instructions causing the processor to execute:
training a prediction model based on actual data comprising a plurality of explanatory factors and an objective factor that are related to the polycondensation reaction; and
predicting, with the prediction model, the objective factor during the polycondensation reaction based on the explanatory factors related to the polycondensation reaction, in which
the explanatory factors include a plurality of feature values obtained by a clustering analysis of time-series data from a plurality of measurement instruments at a dehydration temperature rising process, and
the objective factor includes at least either a viscosity or an acid value.

According to the method for performing prediction related to a polycondensation reaction, the information processing device, and the recording medium storing instructions in one or more embodiments of the present disclosure, the prediction technology related to the polycondensation reaction can be improved.

### BRIEF DESCRIPTION OF DRAWINGS

FIG. 1 is a block diagram illustrating the schematic configuration of an information processing device according to one or more embodiments.

FIG. 2 is a flowchart illustrating operations of the information processing device according to one or more embodiments.

FIG. 3 is concept of a polycondensation reaction process according to one or more embodiments.

FIG. 4A is a view illustrating the time transition of each piece of data pertaining to a dehydration temperature rising process.

FIG. 4B illustrates the time transition of categories related to the dehydration temperature rising process.

FIG. 5 illustrates one example of the accuracy verification result of a prediction model according to one or more embodiments.

FIG. 6 illustrates one example of the accuracy verification result of the prediction model according to one or more embodiments.

FIG. 7 is one example of a visualization graph according to one or more embodiments.

FIG. 8 is one example of the conceptual view of a prediction model in one or more embodiments.

### DETAILED DESCRIPTION OF EMBODIMENTS

Hereinafter, a method for performing prediction related to a polycondensation reaction, an information processing device, and a recording medium storing instructions in one or more embodiments of the present disclosure will be described with reference to the drawings. A prediction target according to one or more embodiments of the present disclosure includes both batch reaction and continuous reaction. Examples of the main polymer materials synthesized by the polycondensation reaction according to one or more embodiments include polyesters, polyamides, polyethylene terephthalate, urea resins, phenolic resins, silicone resins, alkyd resins, alkyd resin polyethers, polyglucosides, melamine resins, and polycarbonates. For example, the polycondensation reaction according to one or more embodiments includes a dehydration-condensation reaction of a polyester.

In each of the drawings, the same symbols are assigned to identical or equivalent parts. In the description of one or more embodiments, descriptions of the identical or equivalent parts are omitted or simplified as appropriate.

First, the overview of one or more embodiments will be described. The method for performing prediction related to a polycondensation reaction in one or more embodiments is executed by an information processing device 10. The information processing device 10 trains a prediction model based on actual data including a plurality of explanatory factors and an objective factor related to the polycondensation reaction. The information processing device 10 predicts the objective factor during the polycondensation reaction based on the explanatory factors related to the polycondensation reaction by the trained prediction model. The explanatory factors include a plurality of feature values obtained by clustering analysis of time-series data from a plurality of measurement instruments at a dehydration temperature rising process. The objective factor is characterized by including at least either a viscosity or an acid value.

As described above, according to one or more embodiments, the explanatory factors include the feature values obtained by the clustering analysis of time-series data from the measurement instruments at the dehydration temperature rising process. The objective factor is characterized by including either a viscosity or an acid value. In the case where such an objective factor in the polycondensation reaction is predicted, the prediction accuracy can be improved by including, in the explanatory factors, the feature values obtained by the clustering analysis of the time-series data from the measurement instruments at the dehydration temperature rising process as described later. Therefore, according one or more embodiments, the prediction technology related to the polycondensation reaction can be improved.

### (Configuration of Information Processing Device)

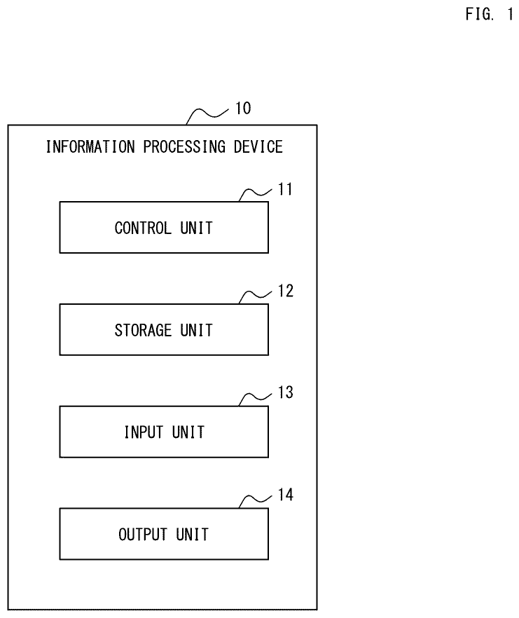

Subsequently, referring to FIG. 1, each configuration of the information processing device 10 will be described in detail. The information processing device 10 is an arbitrary device used by users. For example, personal computers, server computers, general-purpose electronic devices, or dedicated electronic devices can be employed as the information processing device 10.

As illustrated in FIG. 1, the information processing device 10 includes a control unit (or a processor) 11, a storage unit (or a storage) 12, an input unit (or an input interface) 13, and an output unit (or an output interface) 14.

The control unit 11 includes at least one processor, at least one dedicated circuit, or a combination thereof. The processor is a general-purpose processor such as a central processing unit (CPU) or a graphics processing unit (GPU), or a dedicated processor specialized for specific processing. The dedicated circuit is, for example, a field-programmable gate array (FPGA) or an application specific integrated circuit (ASIC). The control unit 11 executes processes associated with the operation of the information processing device 10 while controlling each part of the information processing device 10.

The storage unit 12 includes at least one semiconductor memory, at least one magnetic memory, at least one optical memory, or a combination of at least two of these memories. The semiconductor memory is, for example, a random-access memory (RAM) or a read-only memory (ROM). The RAM is, for example, a static random-access memory (SRAM) or a dynamic random-access memory (DRAM). The ROM is, for example, an electrically erasable programmable read-only memory (EEPROM). The storage unit 12 functions, for example, as a main memory device, an auxiliary memory device, or a cache memory. In the storage unit 12, data used in the operation of the information processing device 10 and data obtained by the operation of the information processing device 10 are stored.

The input unit 13 includes at least one interface for input. The interface for input is, for example, physical keys, capacitive keys, pointing devices, or touch screens integrated with displays. The interface for input may be, for example, a microphone that accepts voice input or a camera that accepts gesture input. The input unit 13 accepts operations to input data used in the operation of the information processing device 10. The input unit 13 may be connected to the information processing device 10 as an external input device instead of being provided in the information processing device 10. For example, any method such as universal serial bus (USB), high-definition multimedia interface (HDMI) (registered trademark), or Bluetooth (registered trademark) can be used as the connection method.

The output unit 14 includes at least one interface for output. The interface for output is, for example, a display that outputs information in the form of images. The display is, for example, a liquid crystal display (LCD) or an organic electroluminescence (EL) display. The output unit 14 displays and outputs data obtained by the operation of the information processing device 10. The output unit 14 may be connected to the information processing device 10 as an external output device instead of being provided in the information processing device 10. For example, any method such as USB, HDMI (registered trademark), or Bluetooth (registered trademark) can be used as the connection method.

The functions of the information processing device 10 are achieved by executing a program or instructions according to one or more embodiments on a processor corresponding to the information processing device 10. In other words, the functions of the information processing device 10 are achieved by software. The instructions cause the computer to function as the information processing device 10 by causing the computer to execute the operations of the information processing device 10. In other words, the computer functions as the information processing device 10 by executing the operation of the information processing device 10 in accordance with the instructions.

In one or more embodiments, the instructions can be recorded on computer-readable recording media. The computer-readable recording media include non-transient computer-readable media, for example, magnetic recording devices, optical discs, magneto-optical recording media, or semiconductor memories.

Some or all of the functions of the information processing device 10 may be achieved by a dedicated circuit corresponding to the control unit 11. In other words, some or all of the functions of the information processing device 10 may be achieved by hardware.

In one or more embodiments, the storage unit 12 stores therein, for example, actual data and prediction models. The actual data and the prediction model may be stored in an external device separate from the information processing device 10. In this case, the information processing device 10 may be equipped with an interface for external communication. The interface for communication may be either an interface of a wired communication or an interface of wireless communication. In the case of the wired communication, the interface for communication is, for example, a LAN interface or USB. In the case of the wireless communication, the interface for communication is, for example, an interface compliant with mobile communication standards such as LTE, 4G, or 5G, or an interface compliant with short-range wireless communication such as Bluetooth (registered trademark). The interface for communication can receive data used in the operation of the information processing device 10 and can transmit data obtained by the operation of the information processing device 10.

### (Operation of Information Processing Device)

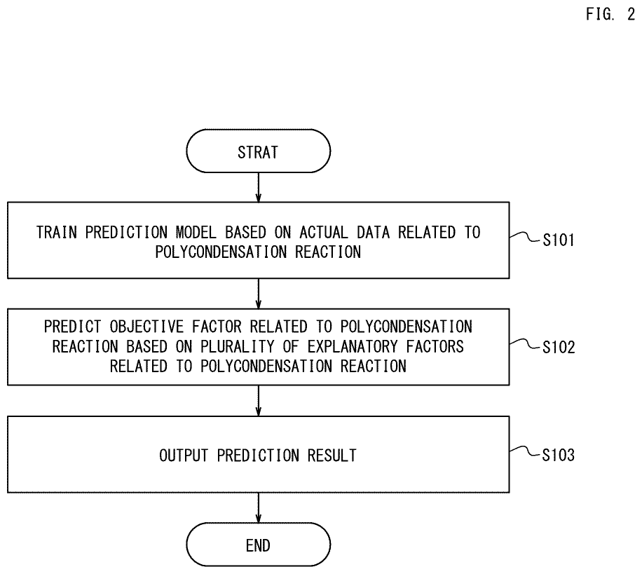

Subsequently, with reference to FIG. 2, the operation of the information processing device 10 according to one or more embodiments will be described.

Step S101: The control unit 11 of the information processing device 10 trains a prediction model based on actual data on the polycondensation reaction. The actual data include the explanatory factors and the objective factor related to the polycondensation reaction. The explanatory factors include the feature values obtained by the clustering analysis of the time-series data from the measurement instruments at the dehydration temperature rising process. The objective factor includes at least either a viscosity or an acid value. In other words, the control unit 11 trains the prediction model using these explanatory factors and objective factor included in the actual data as learning data.

Any method can be employed to acquire the actual data. For example, the control unit 11 acquires the actual data from the storage unit 12. The control unit 11 may also acquire the actual data by accepting input of the actual data from the user by the input unit 13. Alternatively, the control unit 11 may acquire such actual data from an external device that stores therein the actual data through an interface for communication.

The prediction model trained based on the learning data is cross-validated. As a result of such cross-validation, in the case where an accuracy is within a practical range, the prediction related to the polycondensation reaction is performed using the prediction model.

Step S102: The control unit 11 predicts the objective factor related to the polycondensation reaction based on the explanatory factors related to the polycondensation reaction. For example, the control unit 11 may acquire the explanatory factors by accepting input of the explanatory factors from the user by the input unit 13.

Step S103: The control unit 11 outputs the objective factor predicted at Step S102 as the prediction result from the output unit 14.

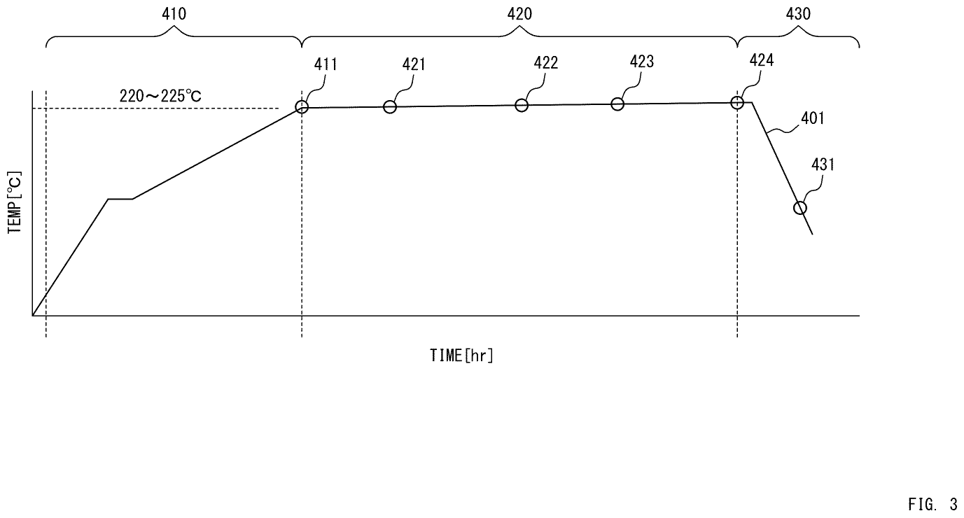

Here, in one or more embodiments, the explanatory factors are characterized by including the feature values obtained by the clustering analysis of the time-series data from the measurement instruments at the dehydration temperature rising process. FIG. 3 is a conceptual view illustrating the polycondensation reaction process. As illustrated in FIG. 3, the polycondensation reaction includes a dehydration temperature rising process 410, a holding process 420, and a cooling process 430. A graph 401 illustrates the temperature transition of a material to be synthesized. At the dehydration temperature rising process 410, the temperature of the material to be synthesized rises. In the holding process 420, the temperature of the material to be synthesized is kept constant. At intermediate stages 421-424 of the holding process 420, the quality values of the material during the reaction are sampled and analyzed by hand. Such quality values include at least either a viscosity or an acid value. The quality values may include a hydroxyl value and physical property values of color. Performing the sampling and the hand analysis at the intermediate stages 421-424 allows the time length of the holding process 420 to be adjusted. At a final stage 431 of the cooling process 430, the quality values of the material are analyzed.

The above analytical values in the polycondensation reaction correspond to the objective factor in one or more embodiments. Such analytical values also depend on the dehydration temperature rising process. On the other hand, a wide variety of time-series data are involved at the dehydration temperature rising process, and thus using all of these time-series data as the explanatory variables is not realistic. Here, the time-series data include measurement values at intervals of 1 second to 1 minute. Therefore, the one or more embodiments are characterized by using the feature values obtained by the clustering analysis of the time-series data from the measurement instruments at the dehydration temperature rising process as the explanatory factors.

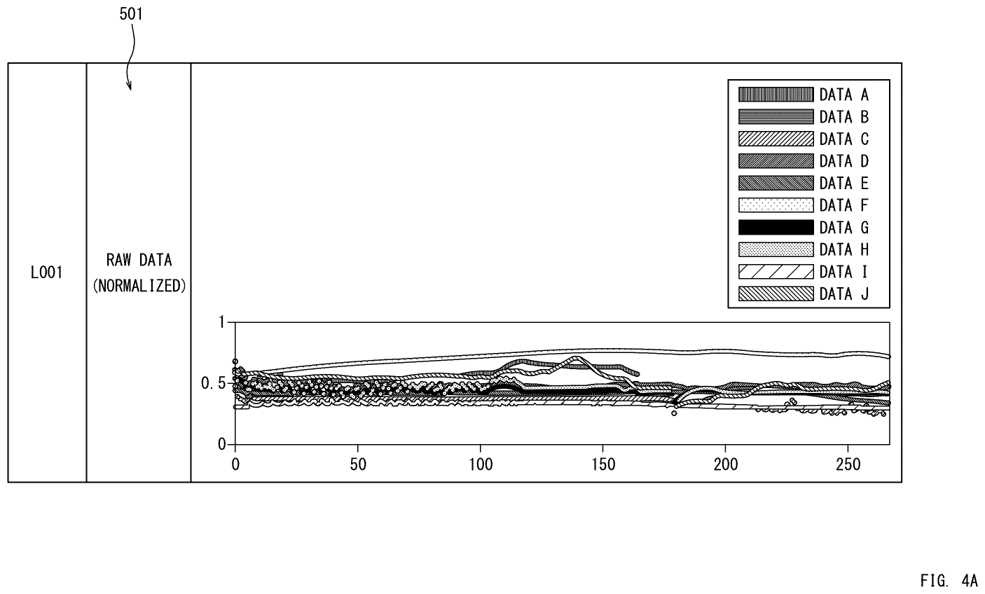

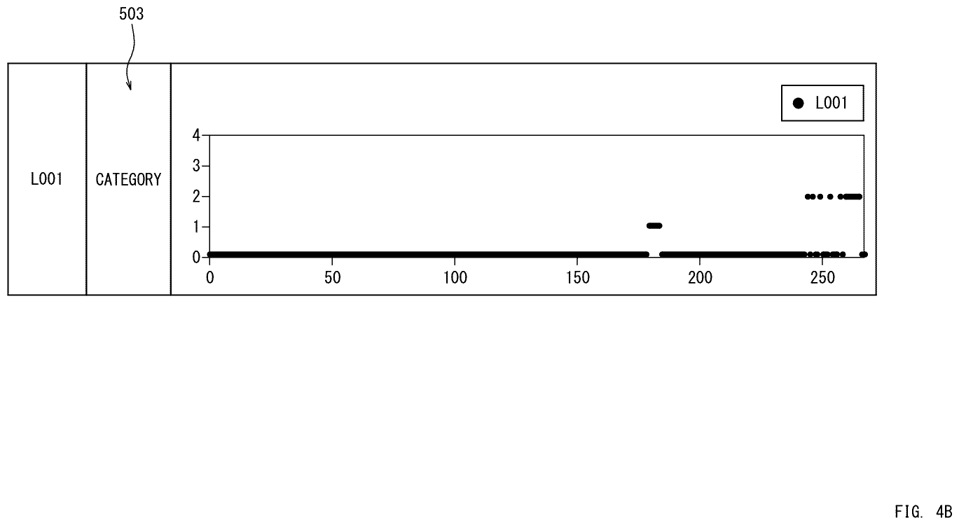

FIG. 4A and FIG. 4B illustrate a method for calculating the feature values in a certain lot (a lot having a lot number of L001). An item 501 in FIG. 4A represents the time transition of each piece of the data pertaining to the dehydration temperature rising process related to such a lot. Examples of such data include data pertaining to a vessel temperature, degasification, a column-top temperature, a reflux amount, a partial condenser inlet/outlet temperature, a heat medium, a vessel pressure, and a gas phase temperature. In FIG. 4A, the data pertaining to the dehydration temperature rising process are represented by Data A to Data J. Data A is data (° C./min) on the gradient of the vessel temperature. Data B is data (kg/h/min) on the gradient of a degassing volume. Data C is data (° C.) on the column-top temperature. Data D is data (kg/h/min) on the gradient of the reflux amount (kg/h/min). Data E is data (° C.) on the inlet temperature to the partial condenser. Data F is data (° C.) on the outlet temperature from the partial condenser. Data G is data (° C./min) on the gradient of the inlet temperature to the heat medium. Data H is data (° C./min) on the gradient of the return temperature to the heat medium. Data I is data (MPa) on the vessel pressure. Data J is data (° C./min) on the gradient of the gas phase temperature. Each pieces of the data in the item 501 is normalized. An item 503 in FIG. 4B represents the time transition of the categories related to the dehydration temperature rising process. In one or more embodiments, the categories include five steps of 0-4. Specifically, the categories are determined by the clustering analysis of the time-series data from the measurement instruments at the dehydration temperature rising process. Based on these categories, the feature values in the dehydration temperature rising process are determined. For example, the feature values are determined based on the time rate of the categories. The time rate of the categories refers to a value obtained by dividing the cumulative residence time of each category by the overall time. As described above, in one or more embodiments, the feature values are provided by the clustering analysis in the dehydration temperature rising process.

As described above, according to one or more embodiments, the explanatory factors include the feature values obtained by the clustering analysis of the time-series data from the measurement instruments at the dehydration temperature rising process. The objective factor is characterized by including either a viscosity or an acid value. In the case where the prediction related to the polycondensation reaction is performed, the accuracy of the prediction model can be improved by including, in the explanatory factors, the feature values obtained by the clustering analysis of the dehydration temperature rising process. Therefore, according one or more embodiments, the prediction technology related to the polycondensation reaction can be improved.

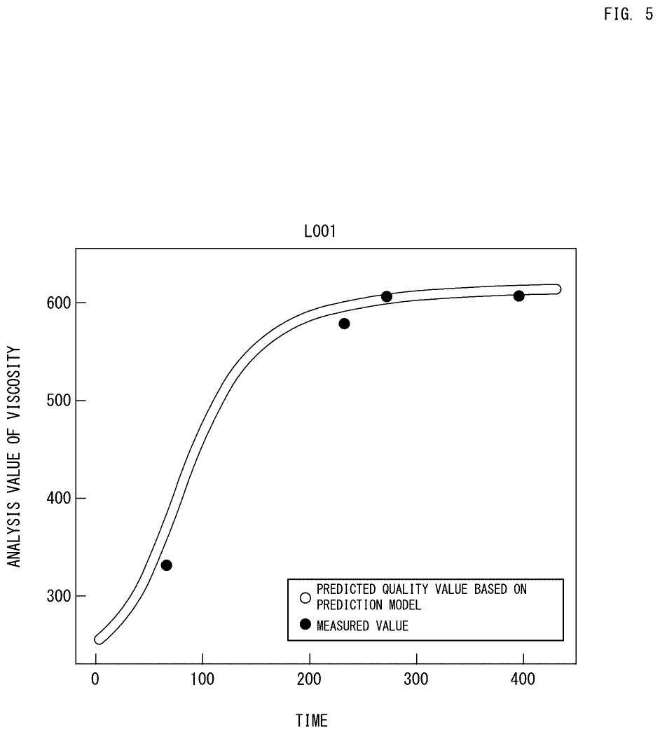

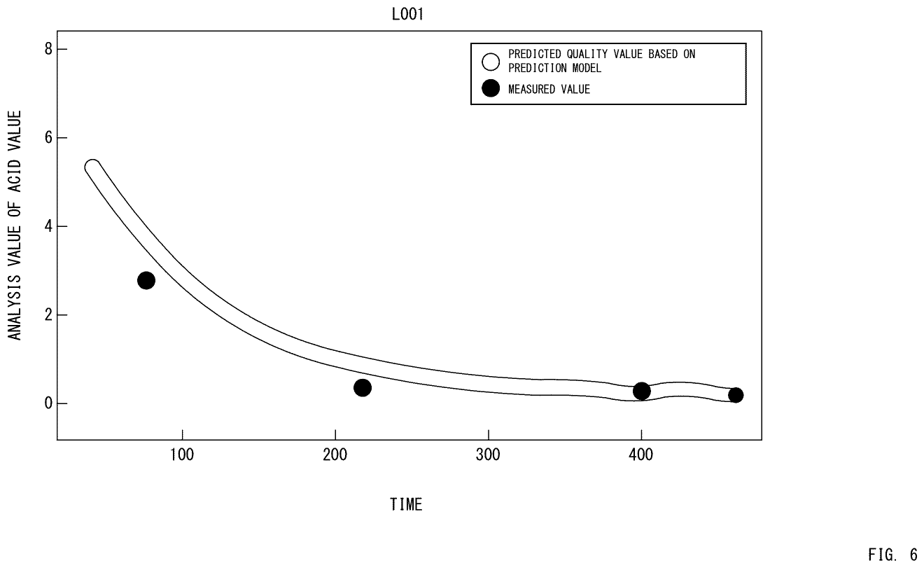

FIG. 5 and FIG. 6 illustrate an example of the accuracy verification results of the prediction model according to one or more embodiments. FIG. 5 is a graph illustrating the viscosity of a certain lot (lot number L001) of polyester predicted by the prediction model and the measured values. As illustrated in FIG. 5, the viscosity predicted by the prediction model approximately agrees with the measured values. The prediction accuracy of the viscosity is ±1.85% relative to a standard value. FIG. 6 is a graph illustrating the acid value of the above lot of polyester predicted by the prediction model and the measured values. As illustrated in FIG. 6, the acid value predicted by the prediction model approximately agrees with the measured values. The prediction accuracy of the acid values is 0.085 in an absolute value relative to a standard value of 0.2 or less. Therefore, it is found that the accuracy of the prediction model is sufficiently high.

Here, the explanatory factors may include theoretical values in a reaction physics model. As described above, the reaction physics model may serve as explanatory factors as a baseline of the reaction characteristics. Similarly, the explanatory factors may include a heat medium return temperature, a vessel temperature, a nitrogen form, a yield, and a nitrogen amount.

In one or more embodiments, a visualization graph may be output by previously predicting the change over time of the objective factor during the polycondensation reaction. Specifically, at the end point of the dehydration temperature rising process 410 (a dehydration temperature rising process end point 411), the continuous reaction progress to the reaction end point (before cooling) is previously predicted. Specifically, in this case, the explanatory factors include the cumulative calculation value of an added amount of a raw material (cumulative calculation value of raw material addition) and the raw material addition time during the polycondensation reaction. Then, the cumulative calculation value of raw material addition and the addition time thereof, which are some of conditions for calculating the predicted value in the change over time of the objective factor, are changed, whereby the visualization graph illustrating the relationship among a reaction end time, the amount of the added raw material, and the raw material addition time is output. Here, the reaction end time refers to a time until the physical property values of the material reach the target values. The amount of the added raw material refers to the added amount of the raw material that allows the product satisfying product specifications to be prepared in a single adjustment charge.

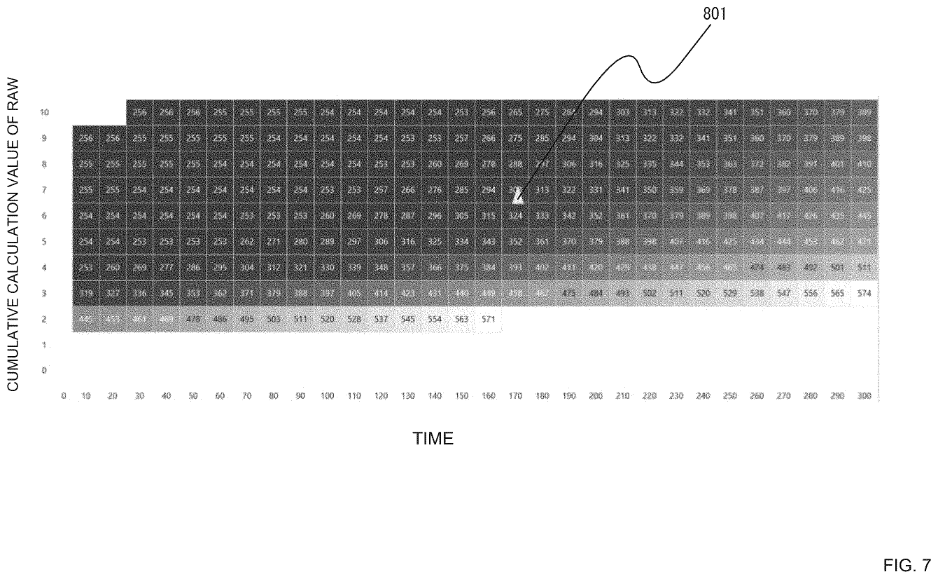

Such a visualization graph is an arbitrary graph in which a first axis is the raw material addition time and a second axis is the amount of the added raw material. For example, the visualization graph includes a heat map and a contour map. FIG. 7 illustrates one example of the visualization graph. FIG. 7 is one example in the case where the visualization graph is a heat map. In the heat map of FIG. 7, the horizontal axis corresponds to the first axis and represents the raw material addition time. The vertical axis of the heat map corresponds to the second axis and represents the amount of the added raw material. Each cell may indicate the numerical value of the reaction end time. In such a heat map, the shade of each cell changes depending on the reaction end time. Specifically, as the reaction end time becomes smaller, the shade of the cell becomes darker. This visualizes the relationship among the addition time of the added raw material, the amount of the added raw material, and the reaction end time. In other words, the addition time of the added raw material and the amount of the added raw material can be easily visually grasped in order to achieve the shortest possible reaction end time by the visualization graph such as the heat map.

Here, the visualization graph may include plots representing actual data. A plot 801 in FIG. 7 represents the actual data determining the added amount of the added raw material to be 7 and the addition time of the added raw material to be 170. The plot 801 of the actual data allows the optimal addition time and added amount of the added raw material to be determined by comparing and considering such actual data.

Here, the prediction model according to one or more embodiments may be, for example, a neural network model. In the case where the prediction model is the neural network model, the coefficients of activation functions of the neural network model may be different between an intermediate layer and an output layer. For example, the coefficient of the activation function of the intermediate layer is larger than the coefficient of the activation function of the output layer.

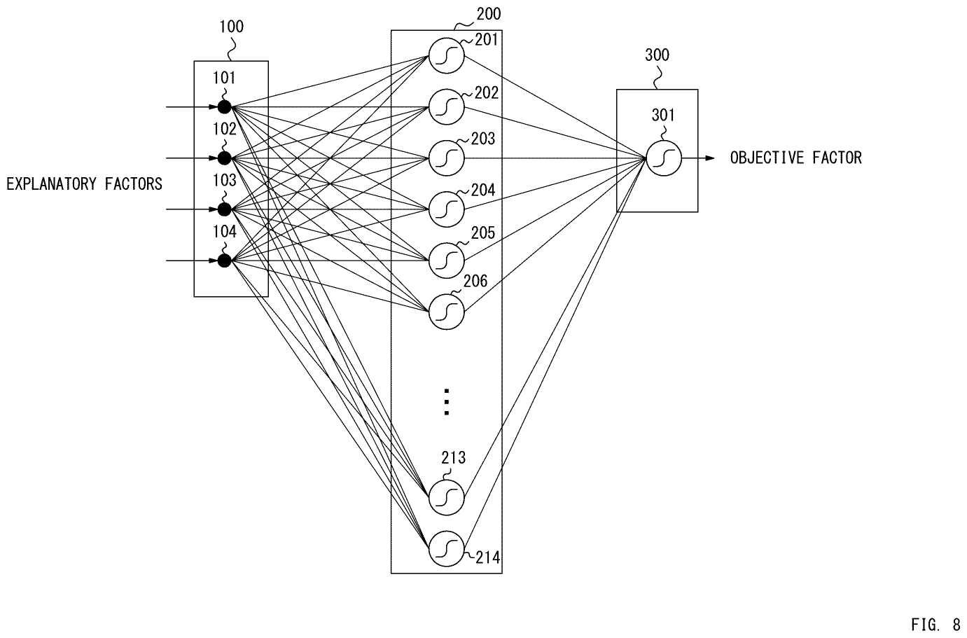

FIG. 8 is a conceptual view of the neural network model according to one or more embodiments. Such a neural network model includes an input layer 100, an intermediate layer 200, and an output layer 300. The neural network model in one or more embodiments is fully connected. In one or more embodiments, the number of layers in the neural network model is, for example, 2. Such number of layers is the number of layers excluding the input layer. By setting the number of layers in the neural network model to 2, a model configuration can be prevented from becoming inappropriate to the physical phenomena in the polycondensation reaction. In other words, the number of layers of neural network model can be kept to minimum necessary, whereby the model configuration suitable for the physical phenomena in the polycondensation reaction can be achieved. The number of layers of the neural network model according to one or more embodiments is not limited to this number of layers, and may be three layers or more. In the case where the number of layers in the neural network model is three or more, as the layer of the neural network model becomes more front side, the coefficient of the activation function may be set larger.

The input layer 100 includes a plurality of elements 101 to 104 (also referred to as input elements 101 to 104). In the neural network model illustrated in FIG. 8, the number of the input elements is 4. The input elements 101 to 104 are also referred to as the first to fourth elements, respectively. In the input elements 101 to 104, each of the explanatory factors is input. The number of input elements is not limited to this and may be less than 4 or 5 or more.

The intermediate layer 200 includes a plurality of elements 201 to 214 (also referred to as intermediate elements 201 to 214). In the neural network model illustrated in FIG. 8, the number of the intermediate elements is 14. The intermediate elements 201 to 214 are also referred to as the first to fourteenth elements, respectively. The number of intermediate elements is not limited to this, and may be less than 14 or 15 or more.

The output layer 300 includes an element 301 (an output element 301). In the neural network model illustrated in FIG. 8, the number of the output elements is 1. The output element 301 is also referred to as the first element The number of the output elements is not limited to this, and may be 2 or more.

The values input from the input elements 101 to 104 of the input layer 100 to the intermediate elements 201 to 214 of the intermediate layer 200 are converted in the intermediate layer 200 based on the activation function of the intermediate layer 200. The converted values are output to the element 301 of the output layer 300. The activation function of the intermediate layer 200 is, for example, a sigmoid function. The values input from the intermediate elements 201 to 214 of the intermediate layer 200 to the output element 301 of the output layer 300 are converted in the output layer 300 based on the activation function of the output layer 300 and output. The activation function of the output layer 300 is, for example, the sigmoid function. Specifically, the activation functions of the intermediate layer and the output layer are, for example, the respective sigmoid functions determined by the following formulas (1) and (2).

$$[ Mathematical Formula 1 ]$$

$${f}^{1} ( {u}_{j}^{1} ) = \frac{1}{1 + {e}^{- {a}_{1} {u}_{j}^{1}}} \qquad ( 1 )$$

$${f}^{2} ( {u}_{j}^{2} ) = \frac{1}{1 + {e}^{- {a}_{2} {u}_{j}^{2}}} \qquad ( 2 )$$

Here, f¹(uⱼ¹) is the activation function of the intermediate layer 200, a₁ is the coefficient of the activation function of the intermediate layer 200, and uⱼ¹ is the input value input to the j-th element of the intermediate layer 200. In the example in FIG. 8, the number of the intermediate elements is 14, and this j takes the value from 1 to 14. f²(uⱼ²) is the activation function of the output layer 300, a₂ is the coefficient of the activation function of the output layer 300, and uⱼ² is the input value input to the j-th element of the output layer 300. In the example in FIG. 8, j is 1 because the number of the output elements is 1. As described above, in the neural network model according to one or more embodiments, the coefficient of the activation function of the intermediate layer 200 is larger than the coefficient of the activation function of the output layer 300. In other words, a₁ and a₂ in the neural network model according to one or more embodiments satisfy a₁>a₂.

In the neural network model according to one or more embodiments, the coefficient of the activation function of the intermediate layer is larger than the coefficient of the activation function of the output layer. This allows the configuration of the neural network model to be optimized at the time of performing the prediction related to the polycondensation reaction. Specifically, in the neural network model for performing the prediction related to the polycondensation reaction, change in the explanatory factors is desirably viewed as obvious change. Therefore, by setting the coefficient of the activation functions of the intermediate layer larger than the coefficient of the activation functions of the output layer, the change in the input values to the intermediate layer can be transmitted to the output layer as the obvious change. On the other hand, in the output layer of the neural network model for performing the prediction related to the polycondensation reaction, the values of the training data and the objective factor are required to be converged. Therefore, the coefficient of the activation function of the output layer is set smaller than the coefficient of the activation function of the intermediate layer. By doing so, the value of the objective factor output from the output layer is finely adjusted.

By setting the coefficients of the activation functions between the intermediate layer and the output layer to be different, the learning process of the neural network model is optimized. Specifically, the updated amounts of the weight variables in the output layer and the intermediate layer during the learning process can be adjusted by changing the coefficient of the activation function. In addition, updating the weight variables provides a significant impact on the learning process. Therefore, the learning process may be optimized based on the adjustment of the updated amounts.

Specifically, in the neural network model at the time of performing prediction related to the polycondensation reaction, the updated amount of the weight variables in the intermediate layer may be set to be relatively large. This allows the weight variables in the intermediate layer to vary more significantly during the learning process, and thus changes in the input values to the intermediate layer to be transferred to the output layer as obvious changes. On the other hand, the updated amount of weight variables in the output layer may be set to be relatively small. This allows the weight variables in the output layer to vary less during the learning process and thus the values of the training data and the objective factor to be easily converged. In addition, by satisfying α1>α2, an arbitrary smooth function can be approximated with sufficient accuracy, eliminating the need to inadvertently increase the number of intermediate layers. This allows sufficient accuracy to be obtained even when the intermediate layer is one layer. Preparing fewer intermediate layers directly leads to reduction in generation of over-fitting and thus provides a secondary effect on stability of the learning process and, in addition, robustness of the model.

The case where the activation functions of the intermediate layer and the output layer are sigmoid functions is described in one or more embodiments. However, the activation functions are not limited to the sigmoid functions. For example, the activation functions of the intermediate layer and the output layer may be functions such as a hyperbolic tangent function (tan h function) and a ramp functions (ReLU).

Although the present disclosure has been described based on the drawings and examples, it should be noted that those skilled in the art can easily make changes and modifications based on the present disclosure. Therefore, it should be noted that these changes and modifications are included within the scope of the present disclosure. For example, the functions and the like included in the units, steps, or the like can be rearranged so as not to be logically inconsistent, and a plurality of units, steps, or the like can be combined to one or divided.

### REFERENCE SIGNS LIST

10 information processing device
11 control unit
12 storage unit
13 input unit
14 output unit
100 input layer
200 intermediate layer
300 output layer
101 to 104, 201 to 214, and 301 element
401 graph
410 dehydration temperature rising process
411 end point of dehydration temperature rising process
420 holding process
421 to 424 intermediate stage
430 cooling process
431 final stage
501 and 503 item
801 plot

1. A method for performing prediction related to a polycondensation reaction, the method being executed by an information processing device and comprising:
training a prediction model based on actual data comprising a plurality of explanatory factors and an objective factor that are related to the polycondensation reaction; and
predicting, with the prediction model, the objective factor during the polycondensation reaction based on the explanatory factors related to the polycondensation reaction, wherein
the explanatory factors include a plurality of feature values obtained by a clustering analysis of time-series data from a plurality of measurement instruments at a dehydration temperature rising process, and
the objective factor includes at least either a viscosity or an acid value.

2. The method according to claim 1, wherein the explanatory factors include a theoretical value in a reaction physics model.

3. The method according to claim 1, wherein the polycondensation reaction is a dehydration-condensation reaction of a polyester.

4. The method according to claim 1, wherein
in the predicting, a time-course change of the objective factor during the polycondensation reaction is previously predicted, and
the explanatory factors include a cumulative calculation value of added raw material during the polycondensation reaction, and a raw material addition time, and
the method further comprises:
changing the cumulative calculation value and the raw material addition time, as a part of a condition for calculating a predicted value of the time-course change of the objective factor, and
outputting a visualization graph illustrating a relationship among a reaction end time, an amount of the added raw material, and the raw material addition time.

5. The method according to claim 4, wherein the visualization graph is a heat map or a contour map in which a first axis indicates the raw material addition time and a second axis indicates the amount of the added raw material.

6. The method according to claim 4, wherein the visualization graph includes plots representing the actual data.

7. The method according to claim 1, wherein
the prediction model is a neural network model including;
an input layer;
an intermediate layer; and
an output layer, and
a coefficient of an activation function of the intermediate layer is larger than a coefficient of an activation function of the output layer.

8. An information processing device performing prediction related to a polycondensation reaction, the information processing device comprising:
a control unit that:
trains a prediction model based on actual data comprising a plurality of explanatory factors and an objective factor that are related to the polycondensation reaction, and
predicts, with the prediction model, the objective factor during the polycondensation reaction based on the explanatory factors related to the polycondensation reaction,
the explanatory factors include a plurality of feature values obtained by a clustering analysis of time-series data from a plurality of measurement instruments at a dehydration temperature rising process, and
the objective factor includes at least either a viscosity or an acid value.

9. A non-transitory computer-readable recording medium storing instructions performing prediction related to a polycondensation reaction by an information processing device that comprises a processor, the instructions causing the processor to execute:
training a prediction model based on actual data comprising a plurality of explanatory factors and an objective factor that are related to the polycondensation reaction; and
predicting, with the prediction model, the objective factor during the polycondensation reaction based on the explanatory factors related to the polycondensation reaction, wherein
the explanatory factors include a plurality of feature values obtained by a clustering analysis of time-series data from a plurality of measurement instruments at a dehydration temperature rising process, and
the objective factor includes at least either a viscosity or an acid value.
